(19) United States
(12) Patent Application Publication
(10) Pub. No.: US 2021/0038330 A1
(43) Pub. Date: Feb. 11, 2021

(54) METHOD AND APPARATUS FOR OPTIMIZING RANGE OF MOTION OF TWO ROBOTIC ARMS IN A ROBOTIC SURGICAL SYSTEM

(71) Applicant: TransEnterix Surgical, Inc., Morrisville, NC (US)

(72) Inventor: Kevin Andrew Hufford, Cary, NC (US)

(21) Appl. No.: 16/932,665

(22) Filed: Jul. 17, 2020

Related U.S. Application Data
(60) Provisional application No. 62/874,986, filed on Jul. 17, 2019.
(60) Provisional application No. 62/874,986, filed on Jul. 17, 2019.

(51) Int. Cl. A61B 34/00; A61B 34/30; B25J 9/00

(52) U.S. Cl. CPC A61B 34/70; B25J 9/0009; B25J 9/0087; A61B 34/30

(57) ABSTRACT

A robotic manipulator system includes a base structure and a pair of manipulator arms on the base structure. Each manipulator arm includes a plurality of arm members, including a base member that includes a central axis. The manipulator arms are arranged on the base structure such that their central axes are collinear.

This application claims the benefit of the following U.S. Provisional Applications: 62/874,986, filed Jul. 17, 2019, and 62/874,987, filed Jul. 17, 2019. Each of these applications is incorporated by reference in its entirety.

### BACKGROUND

Surgical robotic systems are typically comprised of one or more robotic manipulators and a user interface. The robotic manipulators carry surgical instruments or devices used for the surgical procedure. A typical user interface includes input devices, or handles, manually moveable by the surgeon to control movement of the surgical instruments carried by the robotic manipulators. The surgeon uses the interface to provide inputs into the system and the system processes that information to develop output commands for the robotic manipulator.

In the system illustrated in FIG. 1, a surgeon console 12 has two input devices or handles 17, 18. The input devices are configured to be manipulated by a user to generate signals that are used to command motion of a robotically controlled device in multiple degrees of freedom. In use, the user selectively assigns the two input devices to two of the robotic manipulators 13, 14, 15, allowing surgeon control of two of the surgical instruments 10a, 10b, and 10c disposed at the working site at any given time. To control a third one of the instruments disposed at the working site, one of the two input devices is operatively disengaged from one of the initial two instruments and then operatively paired with the third instrument. A fourth robotic manipulator, not shown in FIG. 1, may be optionally provided to support and maneuver an additional instrument.

One of the instruments 10a, 10b, 10c is a camera that captures images of the operative field in the body cavity. The camera may be moved by its corresponding robotic manipulator using input from a variety of types of input devices, including, without limitation, one of the new haptic interface devices, the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices are configured to be manipulated by a user to generate signals that are processed by the system to generate instructions used to command motion of the manipulators in order to move the instruments in multiple degrees of freedom.

In some surgical systems, sensors are used to determine the forces that are being applied to the patient by the robotic surgical tools during use. Such systems make use of force/torques sensor on a surgical robotic manipulator as a method for determining the haptic information needed to provide force feedback to the surgeon at the user interface.

US Patent Publication US 2010/0094312 describes a surgical robotic system in which sensors are used to determine the forces that are being applied to the patient by the robotic surgical tools during use. This application describes the use of a 6 DOF force/torque sensor attached to a surgical robotic manipulator as a method for determining the haptic information needed to provide force feedback to the surgeon at the user interface. The forces are communicated to the surgeon in the form of tactile haptic feedback at the hand controllers of the surgeon console.

For smaller operating rooms, it may be beneficial to reduce the amount of floor spaced used by positioning more than one manipulator on a common base. Doing so, however, can sacrifice range of motion of each manipulator. The following disclosure describes manipulator arm configurations utilizing a reduced amount of floor space by positioning two arms on a common base in a manner that allows a maximal desired range of motion for the manipulator arms.

### BRIEF DESCRIPTION OF THE DRAWINGS

FIG. 1 shows an example of a robot-assisted surgical system;

FIG. 2 illustrates two seven degree-of-freedom robotic arms, mounted on a common base structure, with their base axes collinear.

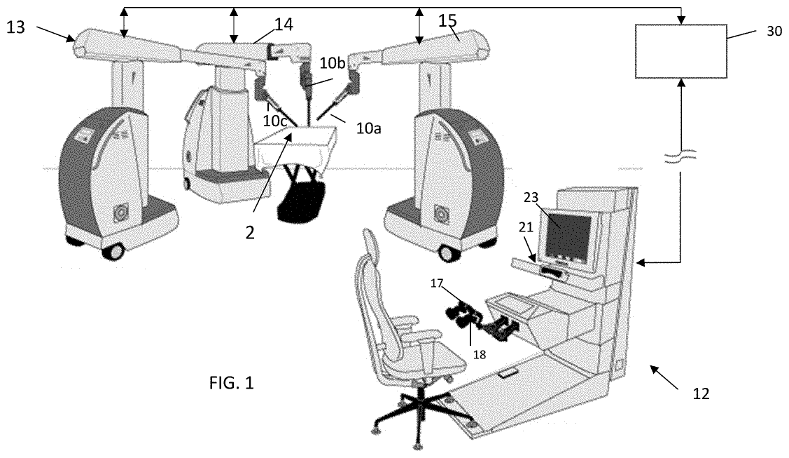

### DETAILED DESCRIPTION

The described invention is configuration of robotic arms (at least two) arranged on a base structure in a manner that allow each to move in a maximal range of motion while minimizing collisions between the arms. The base axis of a robotic arm (in this case, one having seven degrees of freedom) is often largely unrestricted. However, the placement of a second robotic arm in the near vicinity, such as during a robotic surgical procedure can limit the range of motion of that valuable degree of freedom.

In this invention, the bases of the (at least two) robotic arms are mounted on a common base structure so that the bases of the arms share a common base axis. This allows for maximal flexibility of positioning of the robotic arms and their approach angles before and during a surgical procedure.

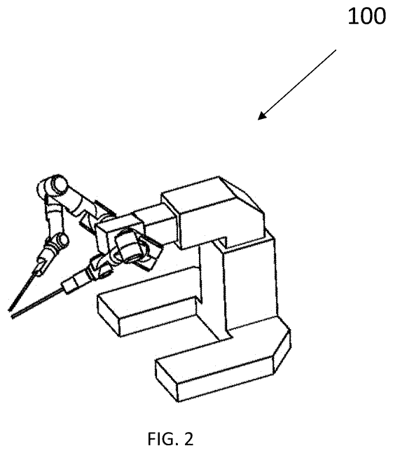

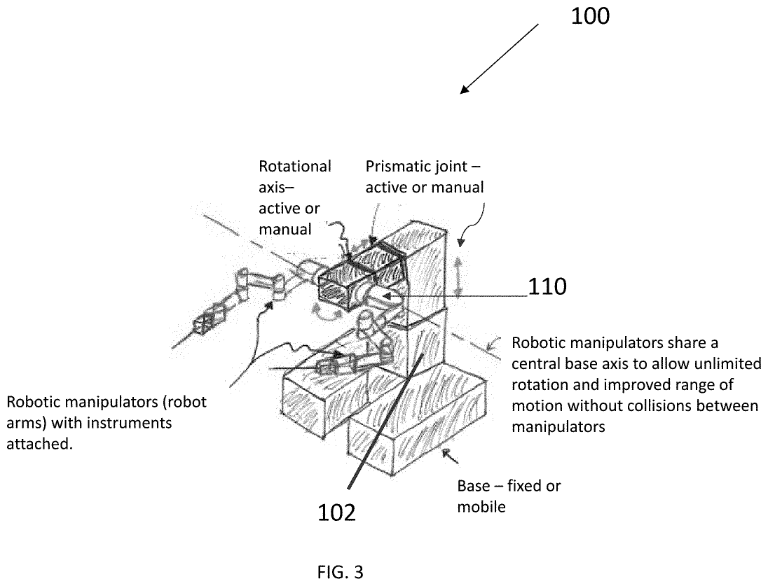

Referring to FIGS. 2 and 3, a robotic manipulator system 100 includes two robotic arms, mounted to a single base structure 102. Each robotic arm has a base element 110 having a base axis. The base axes of each arm are collinear as noted on FIG. 3. The base structure may further comprise additional prismatic and/or rotation degrees of freedom to better position the set of arms with respect to the patient and/or operating table.

In the FIG. 3 embodiment, the final axis of the robotic base to which the two robotic arm bases are attached is a rotational joint. This rotational joint allows easy adjustment of the approach angles with respect to the operating table. This adjustability is especially useful should the Trendelenburg positioning of the patient be adjusted mid-procedure.

Other configurations, with alternate types and or arrangements of degrees of freedom are within the scope of this invention. For instance, in an alternate implementation, there may be a rotational joint just above the first prismatic joint. This may be advantageous for positioning the system's base at an oblique angle to the operating table.

Other configurations having more than two arms are also within the scope of this invention.

The base structure may be one that is fixed or mobile (on wheels, sliders, etc.). In other configurations, the arms or the base structure may instead be bed-mounted, boom-mounted, or ceiling-mounted.

I claim:

1. A robotic manipulator system comprising:
a base structure;
a pair of manipulator arms on the base structure, each manipulator arm having a base member having a central axis, wherein the central axes of each base member are collinear.
